(19) United States
(12) Patent Application Publication
(10) Pub. No.: US 2006/0013795 A1
(43) Pub. Date: Jan. 19, 2006

(54) Composition for hair treatment

(75) Inventors: Emiko Kawata, Yokohama-shi (JP); Tetsuya Kambe, Yokohama-shi (JP)

Correspondence Address: SNIDER & ASSOCIATES, P. O. BOX 27613, WASHINGTON, DC, 20038-7613, US

(73) Assignee: Shiseido Co., ltd., Tokyo (JP)

(21) Appl. No.: 10/518,047

(22) PCT Filed: Jun. 19, 2003
(86) PCT No.: PCT/JP03/07818

§ 371 Date: Jul. 15, 2005

(30) Foreign Application Priority Data
Jun. 20, 2002 (JP) 2002-180217

(51) Int. Cl. A61K 8/46

(52) U.S. Cl. 424/70.24

(57) ABSTRACT

The present invention pertains to a hair treatment composition that contains (a) a specific long-chain acylsulfonate type of anionic surfactant (such as an N-stearoyl-N-methyltaurine salt), (b) a higher alcohol, (c) an anionic polymer, and (d) a reducing agent or an oxidant, in which the combined amount of component (a) and component (b) is 0.5 to 10 weight %, and the molar ratio of component (b) to component (a) is 2 to 10, and provides a hair treatment composition that does not drip off when applied, that is easy to rinse and wash away, that has an excellent feel during and after rinsing, and that does not impair permanent hair treatment effects such as a waving effect, straightening effect, or retexturizing effect.

### TECHNICAL FIELD

This invention relates to a hair treatment composition, and more particularly relates to a hair treatment composition that can be used favorably for permanent waving (“perming”) agents, hair straightening agents used in straight perms and the like, or retexturizing agents which are used on human head hair, such as those that are a cold two-part type, a hot two-part type, a two-part type that heats up when mixed at the time of use, and so forth.

### BACKGROUND ART

A typical method used in the past to give hair a wave or to apply a straight perm was to break up the cystine bonds of hair keratin by using a first agent whose main component was a reducing agent such as a thioglycolate or cysteine, and then reform the cystine bonds with a second agent whose main component was an oxidant such as a bromate or hydrogen peroxide. Because this hair treatment involved oxidation and reduction, the permed hair was prone to damage, dry or rough, and lacked smoothness. Hair that has been damaged, whether by perming or other repeated chemical treatment such as hair coloring or bleaching, or by the physical effects of a dryer or the like, by UV rays, or the like does not feel good to the touch, and instead feels dry or stiff, and also lacks sheen.

When hair in such a damaged state is to be waved, a cationic cream type of first agent has come to be used frequently because it leaves the hair with a softer feel.

However, a cationic cream type of first agent is readily adsorbed by the hair, which means that it takes a long time to rinse out the first agent in the intermediate rinse (when the first agent is washed out with the hair still wound around rods), or the first agent is not completely washed out and hinders the penetration of the second agent into the hair in the next step, which results in problems such as the resulting hair being flat, with no bounce, or the perm being unevenly applied.

A cream base itself adheres well to the hair and is used in perming (both wave and straight types), but if the cream is adjusted to a high enough viscosity that it will not drip off when applied, it takes a long time for the chemical to penetrate into the hair, or the hair absorbs too much of the moisture from the cream while the cream is left on (the cream is left on for a specific time after being applied to the hair), so that the cream dries out and loses most of its original perming effect, or damages the hair.

### DISCLOSURE OF THE INVENTION

It is an object of the present invention to solve the above problems encountered with prior art, and to provide a hair treatment composition that does not drip off when applied, that is easy to rinse out, that has an excellent feel during and after rinsing, and that does not hinder the perming effect, such as the waving effect, straightening effect, or retexturizing effect.

Specifically, to solve the stated problem, the present invention provides a hair treatment composition, comprising (a) a long-chain acylsulfonate type of anionic surfactant expressed by the following General Formula I:
R₁CO—X— (CH₂)ₙSO₃M₁ (I)
(wherein R₁CO— is a saturated or unsaturated fatty acid residue (acyl group) with an average carbon number of 10 to 22; X is —O— or —NR— (wherein R is a hydrogen atom or an alkyl group with a carbon number of 1 to 3); M₁ is a hydrogen atom, an alkali metal, an alkaline earth metal, ammonium, or an organic amine; and n is an integer from 1 to 3), (b) a higher alcohol, (c) an anionic polymer, and (d) a reducing agent or an oxidant, wherein the combined amount of component (a) and component (b) is 0.5 to 10 weight %, and the molar ratio of component (b) to component (a) is 2 to 8.

### BEST MODE FOR CARRYING OUT THE INVENTION

The present invention will now be described in detail.

In the present invention, the long-chain acylsulfonate type of anionic surfactant serving as component (a) is expressed by the following General Formula I:
R₁CO—X— (CH₂)ₙSO₃M₁ (I)

In General Formula I, R₁CO— is a saturated or unsaturated fatty acid residue (acyl group) with an average carbon number of 10 to 22. Examples of R₁CO include C₁₁H₂₃CO, C₁₂H₂₅CO, C₁₃H₂₇CO, C₁₄H₂₉CO, C₁₅H₃₁CO, C₁₆H₃₃CO, C₁₇H₃₅CO, coconut fatty acid residues; and palm fatty acid residues. From standpoints such as safety, it is preferable for R₁CO to have an average carbon number of 12 to 22.

X is —O— or —NR— (wherein R is a hydrogen atom or an alkyl group with a carbon number of 1 to 3). These are electron-donating groups. X is preferably —O—, —NH—, or —N(CH₃)—.

M₁ is a hydrogen atom, an alkaline earth metal, ammonium, or an organic amine. Examples of M₁ include lithium, potassium, sodium, calcium, magnesium, ammonium, monoethanolamine, diethanolamine, triethanolamine, taurine sodium, and N-methyltaurine sodium.

n is an integer from 1 to 3.

Examples of compounds serving as component (a) in which X is —O— in the above General Formula I, that is, of long-chain acylisethionate type anionic surfactants, include cocoyl isethionates, steroyl isethionates, lauryl isethionates, and myristyl isethionates.

Examples of compounds in which X is —NH— in the above General Formula I, that is, of long-chain acyltaurine salt type anionic surfactants, include N-lauroyltaurine salts, N-cocoyl-N-ethanoltaurine salts, N-myristoyltaurine salts, N-stearoyltaurine salts.

Examples of compounds in which X is —N(CH₃)— in the above General Formula I, that is, of long-chain acylmethyltaurine salt type anionic surfactants, include N-lauroyl-N-methyltaurine salts, N-palmitoyl-N-methyltaurine salts, N-steroyl-N-methyltaurine salts, and N-cocoyl-N-methyltaurine salts.

Of these, it is particularly favorable for component (a) to be an N-steroyl-N-methyltaurine salt. Component (a) can be a single compound or a combination of two or more types.

The higher alcohol serving as component (b) is preferably a higher alcohol with a carbon number of 12 to 22 having a linear or branched alkyl chain. Examples of component (b) include lauryl alcohol, cetyl alcohol, stearyl alcohol, behenyl alcohol, myristyl alcohol, oleyl alcohol, cetosteryl alcohol, and other such straight-chain alcohols, and monostearyl glyceryl ether (batyl alcohol), 2-decyltetradecinol, lanolin alcohol, cholesterol, phytosterol, hexyldodecanol, isostearyl alcohol, octyldecanol, and other such branched alcohols. Component (b) can be a single compound or a combination of two or more types.

The combined amount of component (a) and component (b) in the composition of the present invention is 0.5 to 10 weight %. If the combined amount of component (a) and component (b) is less than 0.5 weight %, there will be a problem with dripping when the composition is applied, but if 10 weight % is exceeded the composition will be difficult to rinse out and it will be difficult to obtain a wave with good bounce.

The ratio in which components (a) and (b) are contained is component (b)/component (a)=2 to 10 (molar ratio), and preferable 2 to 8 (molar ratio). If the molar ratio of component (b) to component (a) is less than 2, there will be problems with dripping, but if this molar ratio is over 10 the composition will be difficult to rinse out and it will be difficult to obtain a wave with good bounce.

Examples of the anionic polymer serving as component (c) include sodium polyacrylate, sodium carboxymethyl cellulose, alkyl acrylate/methacrylate copolymers, acrylic resin emulsions, gum arabic, carrageenan, xanthan gum, polyacrylic acid emulsified mixtures, and agar. Of these, a carboxyvinyl polymer is particularly favorable. Commercially available carboxyvinyl polymers include the “Synthalen” series (3-V, made by Sigma of Italy), the “Carbopol” series (made by B.F. Goodrich of the U.S.), and “Hiviswako” (made by Wako Pure Chemicals). Component (c) can consist of a single compound or a combination of two or more types.

The amount in which component (c) is contained in the present invention is preferably 0.05 to 3 weight %, with 0.1 to 2 weight % being particularly favorable. Keeping the component (c) content within the above range facilitates rinsing, which was a drawback to conventional cream formulations containing cationic polymers, and particularly when this composition is used as a first agent, the above range prevents the first agent from remaining on the hair, so the chemical of the second agent can fully penetrate into the hair and the desired perm (such as a bouncy perm) can be obtained more effectively. This also prevents dripping when the composition is applied.

Component (d) is one or more types of reducing agent or oxidant.

There are no particular restrictions on the reducing agent as long as it is one commonly used for permanent waving agents, but examples include thioglycolic acid, thioglycolates, cysteine, cysteine hydrochloride, N-acetylcysteine, thiolactic acid, thiolactates; and other such mercapto compounds, sulfites, thiosulfates, and hydrogensulfites. Of these, thioglycolic acid, thioglycolates, sulfites, cysteine, and so forth are preferable.

There are no particular restrictions on the oxidant as long as it is one commonly used for permanent waving agents, but examples include hydrogen peroxide, alkali metal salts of bromic acid such as sodium bromate or potassium bromate, and alkali metal salts of perboric acid or persulfuric acid such as sodium perborate or sodium persulfate. Particularly, hydrogen peroxide, sodium bromate, potassium bromate, or the like is favorable.

When one or more types of reducing agent are used as component (d), the composition of the present invention is mainly used as a first agent, such as a permanent waving agent or hair straightener (straight perming agent). In this case, the second agent can be any known second agent whose main component is an oxidant. The oxidants listed above can be used favorably as this oxidant, but this list is not, of course, intended to be comprehensive.

When one or more types of reducing agent are thus used as component (d), the amount in which these reducing agents are contained in the composition of the present invention should be 1 to 19 weight %. It is undesirable for the reducing agent content to be too low because the permanent waving effect or hair straightening effect may be inadequate, or for the reducing agent content to be too high because the hair may be damaged.

Meanwhile, when one or more types of oxidant are used as component (d), the composition of the present invention is mainly used as a second agent, such as a hair straightener (straight perming agent). In this case, the first agent can be any known first agent whose main component is a reducing agent. The reducing agents listed above can be used favorably as this reducing agent, but this list is not, of course, intended to be comprehensive. This composition can also be used favorably for retexturizing agents such as kink straighteners or agents that prolong the effect of a straight perm.

When one or more types of oxidant are used as component (d), this oxidant is preferably contained in the composition of the present invention in an amount of 0.6 to 12 weight %. It is undesirable for the oxidant content to be too low because the permanent waving effect or hair straightening effect may be inadequate, or for the oxidant content to be too high because the hair may be damaged.

The composition of the present invention contains components (a) to (c) as essential components in addition to component (d), and if a novel gel base comprising component (a) and component (b) is used as the main component of a perming agent, and component (c) is added to this, the perming effect will be excellent even on damaged hair, an excellent usage feel will be obtained, and the resulting finish will be good.

In addition to the above-mentioned components (a) to (d), various components commonly used in cosmetics, pharmaceuticals, and so on can also be added as desired to the hair treatment composition of the present invention to the extent that the effect of the present invention is not compromised. Examples of such components include oils, silicones, fatty acids, humectants, anionic surfactants other than component (a), nonionic surfactants, amphoteric surfactants, alkalies, acids, metal ion sequestering agents, hydrolyzed proteins, perfumes, and colorants.

There are no particular restrictions on the method for manufacturing the hair treatment composition of the present invention, and any standard method can be employed, but it is preferable to add component (d) and any other added components to components (a) to (c).

The present invention provides a hair treatment composition that does not drip off when applied, that is easy to rinse and wash away, that has an excellent feel during and after rinsing, and that does not impair permanent hair treatment effects such as a waving effect, straightening effect, or retexturizing effect.

### EXAMPLES

The present invention will now be described in further detail through examples, but the present invention is not in any way limited by these examples. All added amounts are given as weight percentages unless otherwise specified.

### Examples 1 to 9, Comparative Examples 1 to 7, Example 10, Comparative Examples 8 to 11

First agents (samples) for permanent waving were prepared from the components listed in Tables 1 to 5 below.

Hair Evaluation Method

Hair (20 cm long, weighing 10 g) which had been bleached for 1 hour was collected from Japanese women in their twenties. This hair was washed and dried and then bundled.

These bundles of hair were evaluated by the following methods by 10 beauticians for ease of rinsing, dripping at application, bounce of wave, and finished feel.

Specifically, each bundle of hair was wound around a rod while the above-mentioned sample (first agent) was worked into the hair in an amount equal to the weight of the hair bundle. Dripping at application was evaluated according to the criteria given below.

The hair was left wound around the rod for 15 minutes, after which it was rinsed with water. The ease of rinsing here was evaluated according to the criteria given below.

The hair was then dried with a towel, after which it was coated with a second agent for permanent waving containing the components listed below, in an amount equal to the weight of the hair bundle, and was left for 15 minutes.

Second Agent for Permanent Waving

| Component | weight % |
|---|---|
| sodium bromate (20%) | 35.0 |
| benzoate | 1.0 |
| buffer (to adjust to pH of 6.5) | as needed |
| nonionic surfactant | 0.5 |
| amino-modified silicone | 1.0 |
| purified water | balance |

The rod was then removed and the hair rinsed with water, and the bounce of the wave and the finished feel after treatment were evaluated according to the criteria given below.

These results are given in Tables 1 to 5.

Ease of Rinsing

Evaluation Criteria
⊚: at least 8 of 10 panelists answered “easy to rinse”
◯: 6 or 7 of 10 panelists answered “easy to rinse”
Δ: 4 or 5 of 10 panelists answered “easy to rinse”
x: 3 or fewer of 10 panelists answered “easy to rinse”
   Dripping

Evaluation Criteria
⊚: at least 8 of 10 panelists answered “no dripping at application”
◯: 6 or 7 of 10 panelists answered “no dripping at application”
Δ: 4 or 5 of 10 panelists answered “no dripping at application”
x: 3 or fewer of 10 panelists answered “no dripping at application”
   Bounce

Evaluation Criteria
⊚: at least 8 of 10 panelists answered “wave had bounce”
◯: 6 or 7 of 10 panelists answered “wave had bounce”
Δ: 4 or 5 of 10 panelists answered “wave had bounce”
x: 3 or fewer of 10 panelists answered “wave had bounce”
   Finished Feel

Evaluation Criteria

⊚: at least 8 of 10 panelists answered “felt good”
◯: 6 or 7 of 10 panelists answered “felt good”
Δ: 0.4 or 5 of 10 panelists answered “felt good”

x: 3 or fewer of 10 panelists answered “felt good”

TABLE 1

| | Example 1 | Example 2 | Example 3 | Example 4 | Example 5 |
|---|---|---|---|---|---|
| ion exchange water | balance | balance | balance | balance | balance |
| Cysteine | 3.5 | 3.5 | 3.5 | 3.5 | 3.5 |
| ammonium thioglycolate | 1.8 | 1.8 | 1.8 | 1.8 | 1.8 |
| (50%) | | | | | |
| ammonium thiolactate | — | — | — | — | — |
| (60%) | | | | | |
| sodium hydrogensulfite | — | — | — | — | — |
| Hydroxyethanediphosphon | 1.0 | 1.0 | 1.0 | 1.0 | 1.0 |
| ic acid (60%) | | | | | |
| alkali (aqueous | as needed | as needed | as needed | as needed | as needed |
| ammonia:monoethanolamine = 1:1) | | | | | |
| (pH adjusted to 9) | | | | | |
| debrominated cetanol | 0.8 | 0.8 | 0.45 | 2.0 | 0.6 |
| behenyl alcohol | — | 2.0 | — | 5.0 | 2.0 |
| sodium | 0.6 | 0.5 | 0.15 | 2.2 | 0.7 |
| steroylmethyltaurine | | | | | |
| anionic polymer | 0.5 | 0.2 | 0.7 | 0.1 | 0.05 |
| (Synthalen K ™) | | | | | |
| nonionic surfactant | 1.0 | 1.0 | 1.0 | 1.0 | 1.0 |
| (Emalex 120 ™) | | | | | |
| ester oil | 2.0 | 2.0 | 2.0 | 2.0 | 2.0 |
| dimethylpolysiloxane | 1.0 | 1.0 | 1.0 | 1.0 | 1.0 |
| (20 cs) | | | | | |
| emulsified silicone | — | — | — | — | — |
| high-molecular weight | — | — | — | — | — |
| silicone | | | | | |
| perfume | as needed | as needed | as needed | as needed | as needed |
| [(b)/(a)] molar ratio | 2.2 | 7.7 | 5.0 | 4.4 | 5.0 |
| [(a) + (b)] content | 1.4 | 3.3 | 0.6 | 9.2 | 3.3 |
| Ease of rinsing | ⊚ | ⊚ | ⊚ | ◯ | ⊚ |
| Dripping at application | ◯ | ⊚ | ⊚ | ⊚ | ◯ |
| Bounce of wave | ⊚ | ⊚ | ⊚ | ◯ | ⊚ |
| Finished feel | ⊚ | ⊚ | ⊚ | ⊚ | ⊚ |

TABLE 2

| | Example | Example | Example | Example |
|---|---|---|---|---|
| | 6 | 7 | 8 | 9 |
| ion exchange water | balance | balance | balance | balance |
| cysteine | 3.5 | — | — | 0.5 |
| ammonium thioglycolate | 1.8 | 13.5 | — | — |
| (50%) | | | | |
| ammonium thiolactate | — | — | 10.0 | — |
| (60%) | | | | |
| sodium hydrogensulfite | — | — | — | 4.0 |
| hydroxyethanedi- | 1.0 | 0.5 | 0.5 | 0.05 |
| phosphonic acid | | | | |
| (60%) | | | | |
| alkali (aqueous | as | as | as | as |
| ammonia:mono- | needed | needed | needed | needed |
| ethanolamine = 1:1) | | | | |
| (pH adjusted to 9) | | | | |
| debrominated cetanol | 0.25 | 0.25 | 0.25 | 1.2 |
| behenyl alcohol | 0.5 | 0.5 | 0.5 | — |
| sodium | 0.2 | 0.2 | 0.2 | 0.5 |
| steroylmethyltaurine | | | | |
| anionic polymer | 1.85 | 0.5 | 0.5 | 0.3 |
| (Synthalen K ™) | | | | |
| nonionic surfactant | 1.0 | 1.0 | 1.0 | 1.5 |
| (Emalex l20 ™) | | | | |
| ester oil | 2.0 | 2.0 | 2.0 | 2.0 |
| dimethylpolysiloxane | 1.0 | 1.0 | 1.0 | 1.0 |
| (20 cs) | | | | |
| emulsified silicone | — | — | 1.0 | — |
| high-molecular weight | — | — | — | 2.0 |
| silicone | | | | |
| perfume | as | as | as | as |
| | needed | needed | needed | needed |
| [(b)/(a)] molar ratio | 5.2 | 5.2 | 5.2 | 4.0 |
| [(a) + (b)] content | 0.95 | 0.95 | 0.95 | 1.7 |
| Ease of rinsing | ◯ | ⊚ | ⊚ | ⊚ |
| Dripping at application | ⊚ | ⊚ | ⊚ | ⊚ |
| Bounce of wave | ◯ | ⊚ | ⊚ | ⊚ |
| Finished feel | ⊚ | ⊚ | ⊚ | ⊚ |

TABLE 3

| | Compar- | Compar- | Compar- | Compar- |
|---|---|---|---|---|
| | ative | ative | ative | ative |
| | Example 1 | Example 2 | Example 3 | Example 4 |
| ion exchange water | balance | balance | balance | balance |
| cysteine | 3.5 | 3.5 | 3.5 | 3.5 |
| ammonium thioglycolate | 1.8 | 1.8 | 1.8 | 1.8 |
| (50%) | | | | |
| hydroxyethane | 1.0 | 1.0 | 1.0 | 1.0 |
| diphosphonate (60%) | | | | |
| alkali (aqueous | as needed | as needed | as needed | as needed |
| ammonia:mono- | | | | |
| ethanolamine = 1:1) | | | | |
| (pH adjusted to 9) | | | | |
| debrominated cetanol | 2.2 | 2.2 | 1.2 | 1.2 |
| behenyl alcohol | — | — | 0.9 | 0.9 |
| stearyltrimethyl- | 0.8 | 0.8 | — | — |
| ammonium chloride | | | | |
| (80%) | | | | |
| behenyltrimethyl- | — | — | 0.7 | 0.7 |
| ammonium chloride | | | | |
| (80%) | | | | |
| cationic polymer | 3.0 | — | 3.0 | — |
| (Merquat 550 ™) | | | | |
| cationic polymer | — | 1.5 | — | 1.5 |
| (Merquat 100 ™) | | | | |
| nonionic surfactant | 1.0 | 1.0 | 1.0 | 1.0 |
| (Emalex 120 ™) | | | | |
| ester oil | 2.0 | 2.0 | 2.0 | 2.0 |
| dimethylpolysiloxane (20 | 1.0 | 1.0 | 1.0 | 1.0 |
| cs) | | | | |
| high-molecular amino- | — | — | — | — |
| modified silicone | | | | |
| perfume | as needed | as needed | as needed | as needed |
| Ease of rinsing | X | X | X | X |
| Dripping at application | X | X | X | X |
| Bounce of wave | Δ | Δ | X | X |
| Finished feel | ⊚ | ⊚ | ⊚ | ⊚ |

TABLE 4

| | Comparative | Comparative | Comparative |
|---|---|---|---|
| | Example 5 | Example 6 | Example 7 |
| ion exchange water | balance | balance | balance |
| cysteine | 3.5 | 3.5 | 5.0 |
| ammonium thioglycolate | 1.8 | 1.8 | 1.8 |
| (50%) | | | |
| hydroxyethane diphosphonate | 1.0 | 1.0 | 1.0 |
| (60%) | | | |
| alkali (aqueous | as needed | as needed | as needed |
| ammonia:monoethanol- | | | |
| amine = 1:1) | | | |
| (pH adjusted to 9) | | | |
| debrominated cetanol | 4.2 | 4.2 | 2.2 |
| behenyl alcohol | 2.0 | 2.0 | — |
| stearyltrimethylammonium | 1.8 | — | 0.8 |
| chloride (80%) | | | |
| behenyltrimethylammonium | — | 1.8 | — |
| chloride (80%) | | | |
| cationic polymer | 2.5 | 2.5 | 3.0 |
| (Merquat 550 ™) | | | |
| cationic polymer | — | — | — |
| (Merquat 100 ™) | | | |
| nonionic surfactant | 1.0 | 1.0 | 1.0 |
| (Emalex 120 ™) | | | |
| ester oil | 2.0 | 2.0 | 2.0 |
| dimethylpolysiloxane (20 cs) | — | — | 1.0 |
| high-molecular amino- | 0.5 | 0.5 | — |
| modified silicone | | | |
| perfume | as needed | as needed | as needed |
| Ease of rinsing | X | X | X |
| Dripping at application | Δ | Δ | X |
| Bounce of wave | X | X | Δ |
| Finished feel | ⊚ | ⊚ | ⊚ |

TABLE 5

| | Comparative | Comparative | Comparative | Comparative | Comparative |
|---|---|---|---|---|---|
| | Example 10 | Example 8 | Example 9 | Example 10 | Example 11 |
| ion exchange water | balance | balance | balance | balance | balance |
| cysteine | 3.5 | 3.5 | 3.5 | 3.5 | 3.5 |
| ammonium thioglycolate | 1.8 | 1.8 | 1.8 | 1.8. | 1.8 |
| (50%) | | | | | |
| hydroxyethane | 1.0 | 1.0 | 1.0 | 1.0 | 1.0 |
| diphosphonate (60%) | | | | | |
| alkali (aqueous | as needed | as needed | as needed | as needed | as needed |
| ammonia:monoethanolamine = | | | | | |
| 1:1) (pH adjusted to 9) | | | | | |
| debrominated cetanol | 0.8 | 0.4 | 0.2 | 3.0 | 2.0 |
| behenyl alcohol | 1.6 | 1.0 | 0.1 | 6.0 | 0.2 |
| sodium | 0.4 | 1.0 | 0.1 | 2.0 | 0.6 |
| steroylmethyltaurine | | | | | |
| anionic polymer | 0.2 | 0.2 | 0.2 | 0.2 | — |
| (Synthalen K ™) | | | | | |
| nonionic surfactant | 1.0 | 1.0 | 1.0 | 1.0 | 1.0 |
| (Emalex 120 ™) | | | | | |
| ester oil | 2.0 | 2.0 | 2.0 | 2.0 | 2.0 |
| dimethylpolysiloxane | 1.0 | 1.0 | 1.0 | 1.0 | 1.0 |
| (20 cs) | | | | | |
| perfume | as needed | as needed | as needed | as needed | as needed |
| [(b)/(a)] molar ratio | 8.4 | 1.9 | 4.6 | 6.3 | 5.9 |
| [(a) + (b)] content | 2.8 | 2.4 | 0.4 | 11.0 | 2.8 |
| Ease of rinsing | ⊚ | ⊚ | ⊚ | X | X |
| Dripping at | ⊚ | X | X | ⊚ | X |
| application | | | | | |
| Bounce of wave | ⊚ | ⊚ | ⊚ | Δ | ⊚ |
| Finished feel | ⊚ | ⊚ | ⊚ | ⊚ | ⊚ |

Representative examples are given below of specific methods for preparing the hair treatment composition pertaining to the present invention for Example 1 in Table 1.

Part of the ion exchange water and 0.5 g of Synthalen K™ were agitated at room temperature (20 to 25° C.) (this mixture will hereinafter be referred to as A). Part of the ion exchange water, 1.8 g of ammonium thioglycolate (50%), 1.0 g of hydroxyethanediphosphonic acid (60%), and 3.5 g of cysteine were agitated and mixed at room temperature (20 to 25° C.) (this mixture will hereinafter be referred to as B).

Next, 0.8 g of debrominated cetanol, 0.6 g of sodium steroylmethyltaurine, 1.0 g of Emalex 120™, 2.0 g of ester oil, and 1.0 g of dimethylpolysiloxane (20 cs) were melted at 80 to 85° C. and then mixed at 70° C. (this mixture will hereinafter be referred to as C). C was gently added to the A prepared previously, and the system was emulsified at low speed in a homomixer. After this composition was cooled to 35° C., B was added, then a suitable amount of perfume was added, and a suitable amount of alkali (ammonia: monoethanolamine=1:1) was added to adjust the pH to 9. The system was agitated at high speed in a homomixer under reduced pressure and at 30 to 32° C., and then deaerated to obtain the targeted composition.

### Example 11

### Straight Perming Agent; First Agent

| Component | weight % |
|---|---|
| ion exchange water | balance |
| ammonium thioglycolate (50%) | 13.0 |
| hydroxyethandiphosphonic acid (60%) | 1.0 |
| alkali (aqueous ammonia:monoethanolamine = 1:1) | as needed |
| (to adjust to pH of 9) | |
| sodium steroylmethyltaurine | 1.0 |
| debrominated cetanol | 1.2 |
| behenyl alcohol | 2.8 |
| nonionic surfactant (Emalex 120 ™) | 1.0 |
| ester oil | 3.0 |
| anionic surfactant (Synthalen K ™) | 0.8 |
| dimethylpolysiloxane (20 cs) | 2.0 |
| dipropylene glycol | 1.0 |
| propylene glycol | 1.0 |
| soy extract | 0.1 |
| hydrolyzed soy | 0.1 |
| perfume | as needed |

The combined amount of component (a) and component (b) in the composition of Example 11 was 5.0 weight %, and the molar ratio of component (b) to component (a) was 5.5.

### Example 12

### Straight Perming Agent; First Agent

| Component | weight % |
|---|---|
| ion exchange water | balance |
| ammonium thioglycolate (50%) | 6.5 |
| monoethanolamine thioglycolate (50%) | 6.5 |
| ammonium dithioglycolate (40%) | 1.0 |
| hydroxyethandiphosphonic acid (60%) | 1.0 |
| alkali (aqueous ammonia:monoethanolamine = 1:1) | as needed |
| (to adjust to pH of 9) | |
| sodium steroylmethyltaurine | 1.0 |
| debrominated cetanol | 1.2 |
| behenyl alcohol | 2.8 |
| nonionic surfactant (Emalex 120 ™) | 1.0 |
| ester oil | 3.0 |
| anionic surfactant (Synthalen K ™) | 0.7 |
| dimethylpolysiloxane (20 cs) | 2.0 |
| dipropylene glycol | 3.0 |
| hydrolyzed keratin | 0.1 |
| hydrolyzed collagen | 0.1 |
| perfume | as needed |

The combined amount of component (a) and component (b) in the composition of Example 12 was 5.0 weight %, and the molar ratio of component (b) to component (a) was 5.5.

### Example 13

### Straight Perming Agent; First Agent

| Component | weight % |
|---|---|
| ion exchange water | balance |
| ammonium thioglycolate (50%) | 20.0 |
| ammonium dithioglycolate (40%) | 8.0 |
| hydroxyethandiphosphonic acid (60%) | 1.0 |
| alkali (aqueous ammonia:monoethanolamine = 1:1) | as needed |
| (to adjust to pH of 9) | |
| sodium steroylmethyltaurine | 1.5 |
| debrominated cetanol | 2.0 |
| behenyl alcohol | 3.5 |
| nonionic surfactant (Emalex 120 ™) | 1.0 |
| ester oil | 3.0 |
| anionic surfactant (Synthalen K ™) | 0.6 |
| aminopropyldimethicone | 1.0 |
| dimethicone | 1.0 |
| dynamite glycerine | 6.0 |
| hydrolyzed quaternary keratin | 0.1 |
| hydrolyzed quaternary collagen | 0.1 |
| perfume | as needed |

The combined amount of component (a) and component (b) in the composition of Example 13 was 7.0 weight %, and the molar ratio of component (b) to component (a) was 5.2.

### Example 14

### Straight Perming Agent; First Agent

| Component | weight % |
|---|---|
| ion exchange water | balance |
| ammonium thioglycolate (50%) | 10.0 |
| monoethanolamine thioglycolate (50%) | 10.0 |
| ammonium dithioglycolate (40%) | 8.0 |
| pentasodium diethylenetriaminepentaacetate | 1.0 |
| alkali (aqueous ammonia:monoethanolamine = 1:1) | as needed |
| (to adjust to pH of 9) | |
| sodium steroylmethyltaurine | 1.5 |
| debrominated cetanol | 2.0 |
| behenyl alcohol | 3.5 |
| nonionic surfactant (Emalex 120 ™) | 1.0 |
| ester oil | 3.0 |
| anionic surfactant (Synthalen K ™) | 0.5 |
| aminopropyldimethicone | 2.0 |
| dimethicone | 2.0 |
| dynamite glycerine | 10.0 |
| hydrolyzed silk | 0.1 |
| hydrolyzed wheat | 0.1 |
| perfume | as needed |

The combined amount of component (a) and component (b) in the composition of Example 14 was 7.0 weight %, and the molar ratio of component (b) to component (a) was 5.2.

### Example 15

### Straight Perming Agent; First Agent

| Component | weight % |
|---|---|
| ion exchange water | balance |
| cysteine | 1.5 |
| monoethanolamine thioglycolate (50%) | 18.0 |
| ammonium dithioglycolate (40%) | 6.0 |
| pentasodium diethylenetriaminepentaacetate | 1.0 |
| alkali (aqueous ammonia:monoethanolamine = 1:1) | as needed |
| (to adjust to pH of 9) | |
| sodium steroylmethyltaurine | 1.5 |
| debrominated cetanol | 2.0 |
| behenyl alcohol | 3.5 |
| nonionic surfactant (Emalex 120 ™) | 1.0 |
| ester oil | 3.0 |
| anionic surfactant (Synthalen K ™) | 0.4 |
| aminopropyldimethicone | 2.0 |
| dimethicone | 2.0 |
| methylsiloxane emulsion | 1.0 |
| dynamite glycerine | 5.0 |
| dipropylene glycol | 3.0 |
| hydrolyzed quaternary silk | 0.1 |
| hydrolyzed quaternary wheat | 0.1 |
| perfume | as needed |

The combined amount of component (a) and component (b) in the composition of Example 15 was 7.0 weight %, and the molar ratio of component (b) to component (a) was 5.2.

### Example 16

### Straight Perming Agent; First Agent

| Component | weight % |
|---|---|
| ion exchange water | balance |
| ammonium thiolactate (50%) | 9.0 |
| monoethanolamine thiolactate (50%) | 9.0 |
| pentasodium diethylenetriaminepentaacetate | 1.0 |
| alkali (aqueous ammonia:monoethanolamine = 1:1) | as needed |
| (to adjust to pH of 9) | |
| sodium steroylmethyltaurine | 1.0 |
| debrominated cetanol | 1.2 |
| behenyl alcohol | 2.8 |
| nonionic surfactant (Emalex 120 ™) | 1.0 |
| ester oil | 3.0 |
| anionic surfactant (Synthalen K ™) | 0.6 |
| dimethylpolysiloxane (20 cs) | 2.0 |
| dynamite glycerine | 2.0 |
| dipropylene glycol | 2.0 |
| honey extract | 0.1 |
| royal jelly | 0.1 |
| perfume | as needed |

The combined amount of component (a) and component (b) in the composition of Example 16 was 5.0 weight %, and the molar ratio of component (b) to component (a) was 5.5.

### Example 17

### Straight Perming Agent; Second Agent

| Component | weight % |
|---|---|
| ion exchange water | balance |
| sodium bromate | 6.0 |
| phosphoric acid buffer | as needed |
| (to adjust to pH of 6.5) | |
| benzoate | 0.5 |
| hydroxyethandiphosphonic acid (60%) | 0.5 |
| sodium steroylmethyltaurine | 1.2 |
| debrominated cetanol | 3.0 |
| behenyl alcohol | 2.0 |
| nonionic surfactant (Emalex 120 ™) | 1.0 |
| ester oil | 2.5 |
| anionic surfactant (Synthalen K ™) | 0.6 |
| dimethylpolysiloxane (20 cs) | 2.0 |
| aminopropyldimethicone | 4.0 |
| green tea extract | 0.1 |

The combined amount of component (a) and component (b) in the composition of Example 17 was 6.2 weight %, and the molar ratio of component (b) to component (a) was 6.3.

### Example 18

### Straight Perming Agent; Second Agent

| Component | weight % |
|---|---|
| ion exchange water | balance |
| sodium bromate | 10.0 |
| phosphoric acid buffer | as needed |
| (to adjust to pH of 6.5) | |
| benzoate | 0.5 |
| hydroxyethandiphosphonic acid (60%) | 0.5 |
| sodium steroylmethyltaurine | 1.2 |
| debrominated cetanol | 3.0 |
| behenyl alcohol | 2.0 |
| nonionic surfactant (Emalex 120 ™) | 1.0 |
| ester oil | 2.5 |
| anionic surfactant (Synthalen K ™) | 0.6 |
| dimethylpolysiloxane (20 cs) | 2.0 |
| aminopropyldimethicone | 4.0 |
| green tea extract | 0.1 |

### Example 19

### Straight Perming Agent; Second Agent

| Component | weight % |
|---|---|
| ion exchange water | balance |
| hydrogen peroxide (50%) | 5.0 |
| phosphoric acid buffer | as needed |
| (to adjust to pH of 4.0) | |
| pentasodium diethylenetriaminepentaacetate | 0.5 |
| sodium steroylmethyltaurine | 1.8 |
| debrominated cetanol | 7.0 |
| nonionic surfactant (Emalex 120 ™) | 1.0 |
| ester oil | 2.5 |
| anionic surfactant (Synthalen K ™) | 0.8 |
| dimethicone | 2.5 |
| methylsiloxane emulsion | 2.5 |
| dipropylene glycol | 0.1 |
| cyclodextrin | 0.1 |

The combined amount of component (a) and component (b) in the composition of Example 19 was 8.8 weight %, and the molar ratio of component (b) to component (a) was 6.4.

### Example 20

### Straight Perming Agent; Second Agent

| Component | weight % |
|---|---|
| ion exchange water | balance |
| hydrogen peroxide (50%) | 7.0 |
| phosphoric acid buffer | as needed |
| (to adjust to pH of 4.0) | |
| pentasodium diethylenetriaminepentaacetate | 0.5 |
| sodium steroylmethyltaurine | 1.8 |
| debrominated cetanol | 7.0 |
| nonionic surfactant (Emalex 120 ™) | 1.0 |
| ester oil | 2.5 |
| anionic surfactant (Synthalen K ™) | 0.8 |
| dimethicone | 2.5 |
| methylsiloxane emulsion | 2.5 |
| dipropylene glycol | 0.1 |
| cyclodextrin | 0.1 |

The combined amount of component (a) and component (b) in the composition of Example 20 was 8.8 weight %, and the molar ratio of component (b) to component (a) was 6.4.

### INDUSTRIAL APPLICABILITY

As detailed above, the present invention provides a hair treatment composition that does not drip off when applied, that is easy to rinse and wash away, that has an excellent feel during and after rinsing, and that does not impair permanent hair treatment effects such as a waving effect, straightening effect, or retexturizing effect.

1. A hair treatment composition, comprising:
(a) a long-chain acylsulfonate type of anionic surfactant expressed by the following General Formula I:
R₁CO—X—(CH₂)ₙSO₃M₁ (I)
(wherein R₁CO— is a saturated or unsaturated fatty acid residue (acyl group) with an average carbon number of 10 to 22; X is —O— or —NR— (wherein R is a hydrogen atom or an alkyl group with a carbon number of 1 to 3); M₁ is a hydrogen atom, an alkali metal, an alkaline earth metal, ammonium, or an organic amine; and n is an integer from 1 to 3);
(b) a higher alcohol;
(c) an anionic polymer; and
(d) a reducing agent or an oxidant,
wherein the combined amount of component (a) and component (b) is 0.5 to 10 weight %, and the molar ratio of component (b) to component (a) is 2 to 10.

2. The hair treatment composition according to claim 1, wherein component (c) is contained in an amount of 0.05 to 3 weight % for the composition.

3. The hair treatment composition according to claim 1, wherein component (d) is an reducing agent and its combined amount is 1 to 19 weight % for the composition.

4. The hair treatment composition according to claim 1, wherein component (d) is an oxidant and its combined amount is 0.6 to 12 weight % for the composition.

5. The hair treatment composition according to claim 1, which is used in permanent wave agents, hair straightening agents, and retexturizing agents.

6. The hair treatment composition according to claim 2, which is used in permanent wave agents, hair straightening agents, and retexturizing agents.
